(19) United States
(12) Patent Application Publication
(10) Pub. No.: US 2021/0106068 A1
(43) Pub. Date: Apr. 15, 2021

(54) BOXER BRIEF FOR SUPPORTING A REMOVABLE BLADDER

(71) Applicant: Longworth Industries, Inc., Southern Pines, NC (US)

(72) Inventors: Randy D. Black, Pinehurst, NC (US); Houston R. Brady, Pinehurst, NC (US); Cassie E. Brown, Antioch, TN (US); Grace B. Ritter, Asheboro, NC (US)

(73) Assignee: Longworth Industries, Inc., Southern Pines, NC (US)

(21) Appl. No.: 17/066,808

(22) Filed: Oct. 9, 2020

Related U.S. Application Data
(60) Provisional application No. 62/913,385, filed on Oct. 10, 2019.

(51) Int. Cl. A41B 9/00

(52) U.S. Cl. CPC A41B 9/004; A41B 9/001

(57) ABSTRACT

A boxer brief includes first and second side panels and a pouch. The pouch includes a first pouch panel and a second pouch panel. The first pouch panel and the second pouch panel each have a first side edge that is attached to a front edge of the first side panel and a second side edge that is attached to a front edge of the second side panel such that the second pouch panel is disposed over the first pouch panel to define a pocket therebetween. The first pouch panel defines a hole therethrough that is configured to receive genitalia therethrough. The pouch is configured to secure a bladder system within the pocket. The second pouch panel defines a top and bottom opening of the pocket that are each capable of permitting the bladder system to pass therethrough.

### CROSS-REFERENCE TO RELATED APPLICATION

This application claims priority to, and benefit from, U.S. Provisional Patent Application Ser. No. 62/913,385, filed Oct. 10, 2019, the entire contents of which are hereby incorporated by reference.

### BACKGROUND

### 1. Technical Field

The present disclosure relates to undergarments and, more specifically, to a boxer brief that is configured to support a removable bladder.

### 2. Discussion of Related Art

In vehicles it is often not practical to provide restroom facilities due to weight, space, and costs concerns. Operators and sometimes passengers in such vehicles may be subjected to long trips without the availability or desire to take restroom breaks. For example, in general aviation and military aviation, pilots and passengers may be subjected to long flying legs or missions without the availability of restroom facilities. If a pilot or passenger has to urinate, it can be distracting and dangerous, as it diverts attention away from flying the aircraft or tasks to be completed in flight. Moreover, there is a need in other situations to provide devices for absorbing urine from workers, children, incontinent adults, animals, hospital patients, and the like.

Adult diapers, catheters, and other urine collection devices are known in the art for absorbing or collecting urine from humans and animals. These devices suffer from a number of drawbacks known in the art.

For example, so-called adult diapers and the like are known for use with incontinent humans. Similar devices have been used with animals and the like. All are based upon infant diaper technology that uses absorption characteristics of various materials to wick away urine from the body. Despite advances in absorption material technology, the wicking action of such absorbents is not always perfect. As a result, excreted urine may remain in contact with the skin of the user, causing rash or other difficulties. While improved diaper materials tend to do a good job of keeping infants dry, these materials may not perform well with adults, particularly when sitting down, as the pressure from the body tends to force the absorbed urine to the surface. Thus, even with advanced superabsorbent materials and the like, diaper technology still has drawbacks that make it unacceptable for adult use, particularly in aviation and other vehicular applications.

Additionally, an important aspect of any relief system is the ability for a wearer to relax to be able to use the device. This is very important as urination begins from a relaxation process not a contraction process. Further, during removal of some relief systems, leakage can occur which can result in unsanitary or undesirable situations and may cause odor or irritation.

Thus, a need exists in the art for a relief system which may be comfortably worn which will collect and remove urine away from the body without irritating skin or creating discomfort.

### SUMMARY

In an aspect of the present disclosure, a boxer brief includes a first side panel, a second side panel, and a pouch. The first and second side panels each include a front edge. The pouch includes a first pouch panel and a second pouch panel. The first pouch panel has a first edge that is attached to the front edge of the first side panel and a second side edge that is opposite to the first side edge and that is attached to the front edge of the second side panel. The first pouch defines a hole therethrough that is configured to receive genitalia therethrough. The second pouch panel has a first side edge that is attached to the front edge of the first side panel and a second side edge that is opposite the first side edge and that is attached to the front edge of the second side panel such that the second pouch panel is disposed over the first pouch panel to define a pocket between the first and second side panels. The pouch is configured to secure a bladder system within the pocket. The second pouch panel has a top edge that extends between the first and second side edges thereof that is free from the first pouch panel to define a top opening of the pocket and having bottom edge that extends between the first and second side edges thereof that is free from the first pouch panel to define a bottom opening of the pocket opposite the top opening. The pocket is capable of receiving the bladder system through the top opening and capable of having the bladder system removed through the bottom opening.

In embodiments, the front edge of the first side panel, the first side edge of the first pouch panel, and the first side edge of the second pouch panel are attached to one another along a first pouch seam and the front edge of the second side panel, the second side edge of the first pouch panel, and the second side edge of the second pouch panel are attached to one another along a second pouch seam. The first and second pouch seams may be formed of fire or flame retardant thread. The first aside panel, the second side panel, the first pouch panel, and the second pouch panel may be fire or flame retardant. For example, the thread and the panels may be formed of flame retardant material or be treated to be fire or flame retardant.

In some embodiments, the boxer brief includes a back panel that is opposite the pouch. The back panel may include a first side edge and a second side edge opposite the first side edge. The first side panel may have a rear edge that is attached to the first side edge of the back panel and the second side panel may have a rear edge that is attached to the second side edge of the back panel. The boxer brief may include a bottom panel that is attached to at least one edge of the first side panel, the second side panel, the back panel, and the first pouch panel. The boxer brief may include a waistband that is formed by a top portion of the first side panel, the second side panel, the back panel, and the first pouch panel.

In certain embodiments, the pouch includes a third pouch panel that has a first side edge that is attached to the front edge of the first side panel and a second side edge that is attached to the front edge of the second side panel. The hole may pass through the third pouch panel. The third pouch panel may be attached to the first pouch panel about the hole. The first pouch panel may be disposed between the second and third pouch panels. The third pouch panel may include top and bottom edges that each extend between the first and second side edges thereof. The top and bottom edges of the third pouch panel may be free from the first pouch panel.

In particular embodiments, the second pouch panel is formed of a stretchable material. The first side panel, the second side panel, and the fir s pouch panel may be formed of a stretchable material.

In another embodiment of the present disclosure, a relief system includes a boxer brief and a bladder system. The boxer brief includes a first side panel, a second side panel, and a pouch. The first and second side panels each have a front edge. The pouch includes a first pouch panel and a second pouch panel. The first pouch panel has a first side edge that is attached to the front edge of the first side panel and a second side edge that is opposite the first side edge and that is attached to the front edge of the second side panel. The first pouch panel defines a hole therethrough that is configured to receive genitalia therethrough. The second pouch panel has a first side edge that is attached to the front edge of the first side panel and a second side edge that is opposite the first side edge that is attached to the front edge of the second side panel such that the second pouch panel is disposed over the first pouch panel to define a pocket between the first and second side panels. The second pouch panel has a top edge that extends between the first and second side edges thereof that is free from the first pouch panel to define a top opening of the pocket and has a bottom edge that extends between the first and second side edges thereof that is free from the first pouch panel to define a bottom opening of the pocket opposite the top opening. The bladder system that has a receptacle that is received within the pocket. The bottom opening is sized to prevent the receptacle from passing therethrough when the bottom opening is in a relaxed state, the receptacle capable of passing through the top opening and the bottom opening.

In embodiments, the bladder system includes a hole that extends from and is in fluid communication with the receptacle. The hose may pass through the bottom opening of the pocket when the receptacle is received within the pocket. The hose may have a first end that is coupled to the receptacle and a second end that is opposite the first end and configured to releasably couple to a reservoir.

In another embodiment of the present disclosure, a boxer brief includes a waistband, a rear side, a front side, and a pouch. The waistband defines a waist opening at a top of the boxer brief. The waist opening may be configured to receive a waist of a wearer of the boxer brief and to secure the boxer brief to the wearer. The rear side extends downward from the waistband. The front side extends downward from the waistband opposite the rear side. The pouch is on the front side and defines a pocket having a top opening that is adjacent the waistband and a bottom opening that is opposite the top opening. The pouch is configured to retain a receptacle of a bladder system within the pocket. The top opening and the bottom opening capable of allowing the receptacle to pass therethrough.

In embodiments, the pouch includes an outer panel and an inner panel with the pocket being defined between the inner and outer panels. The pouch may further include a middle panel that is disposed between the inner and outer panels. The inner, middle, and outer panels may be attached to one another along pouch seams that extend along opposite sides of each of the inner, middle, and outer panels. The inner and middle panels each define a hole that passes therethrough. The inner and middle panels may be hemmed together about the hole. The outer panel has a top edge and a bottom edge that are free from the middle panel to define the top opening and the bottom opening respectively.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is an outside, front view of a boxer brief provided in accordance with the present disclosure;

FIG. 2 is an inside, front view of a portion of the boxer brief of FIG. 1;

FIG. 3 is an outside, rear view of the boxer brief of FIG. 1;

FIG. 4 is an outside, front view of the boxer brief of FIG. 1 with a portion of a bladder system received within a pouch of the boxer brief;

FIG. 5 is an outside, front view of the boxer brief of FIG. 1 with a receptacle of the bladder system received within the pouch of the boxer brief; and

FIG. 6 is an outside, front view of the boxer brief of FIG. 1 with the receptacle of the bladder system partially removed through a bottom opening of the pouch of the boxer brief.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

As used herein, the term “pilot” refers to any person or wearer of the disclosed boxer brief and is not limited to an aircraft pilot. For example, as used herein the term pilot may include, but is not limited to, an astronaut, a wheelchair user, a long distance driver, an emergency vehicle driver, a racecar driver, a military pilot, a glider pilot, a recreational pilot, a ship pilot, a bed-ridden patient, an incontinence patient, or another wearer of the boxer brief. In some applications, it may be beneficial or required for a relief system to be flame retardant or flame resistant. For example, a military pilot or a racecar driver may be required to wear flame retardant clothing including undergarments.

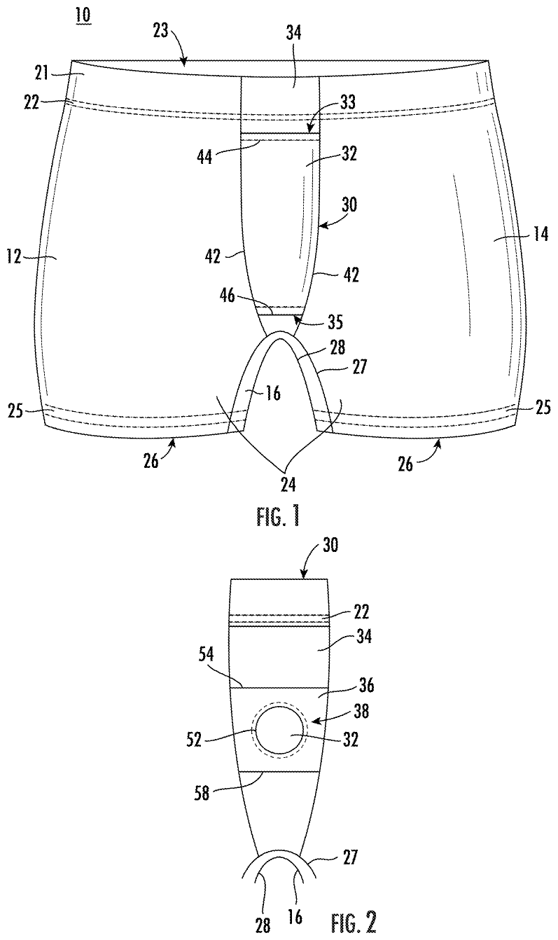

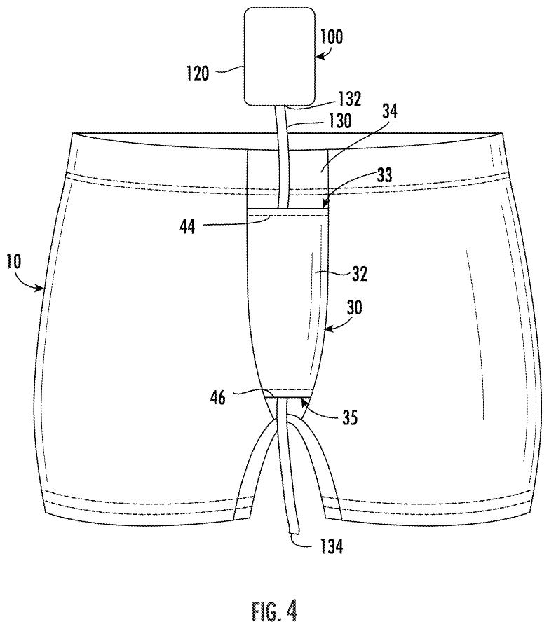

Referring now to FIGS. 1-3, an undergarment that is configured to support a removable bladder is provided in accordance with the present disclosure and shown generally as boxer brief 10. While the undergarment is illustrated as a boxer brief, the undergarment may take another form including, but not limited to, a brief or a bikini brief. The boxer brief 10 has the shape of a traditional boxer brief and includes side panels 12, 14, a bottom or lower panel 16, and a rear panel 18. The boxer brief 10 also includes a pouch 30. The boxer brief 10 includes a waistband 21 that forms an upper portion of each of the side panels 12, 14, back or rear panel 18, and pouch 30. The waistband 21 may include a portion of each of the panels 12, 14, 18 and the pouch 30 that are folded over and secured by a hem 22. The waistband 21 may include a channel that receives a stretchable material such as elastic that is configured to stretch and secure about a waist of a pilot that is received within a waist opening 23 of the boxer brief 10. The elastic may be 1¼ inch elastic that forms a continuous ring within the channel of the waistband 21. The waist opening 23 is configured to receive a waist of a pilot therein and with the waistband 21 secured about the waist of the pilot.

The side panels 12, 14 are each secured to the lower panel 16 by a front lower seam 27 and a rear lower seam 28. The side panels 12, 14 each have a rear edge that is secured to the rear panel 18 by a rear seam 29. The side panels 12, 14 and lower panel 16 form leg portions 24 of the boxer brief 10. Each leg portion 24 may include a hem 25 that extends around a leg opening 26 of the boxer brief 10. The leg opening 26 is configured to allow a leg of a pilot to pass through with the leg portion 24 and grip the leg of the pilot.

The pouch 30 includes an outer panel 32, a middle panel 34, and an inner panel 36. Each of the panels of the pouch 32, 34, 36 includes side edges that are secured to front edges the side panels 12, 14 by a pouch seam 42. The pouch seams 42 are tapered towards one another as the seams 42 extend away from the waist opening 23 towards the lower panel 16. The outer panel 32 is secured to each of the side panels 12, 14 and includes a top hem 44 and a bottom hem 46 that are each free or unattached from the other panels of the pouch 30 along a portion thereof to form a top pouch opening 33 and a lower pouch opening 35 between the outer panel 32 and the middle panel 34. The taper of the pouch seams 42 results in the top pouch opening 33 being larger than the lower pouch opening 35.

As shown, the top pouch opening 33 is positioned below waistband 21, e.g., below the hem 22. In some embodiments, the top pouch opening 33 may be at or above the hem 22 defining the waistband 21. The outer panel 32 may have a width defined between the pouch seams 42 greater than a width of the middle panel 34 such that the pouch 30 may form a pocket between the outer and middle panels 32, 34. The fabric forming the pouch panels 32, 34, 36 may be a resilient or stretchable fabric having a relaxed state and a stretched state. In the relaxed state, the pouch 30 may have a first cross-sectional area at the top hem 44, a second cross-sectional area between the top and bottom hems 44, 46, and a third cross-sectional area at the bottom hem 46. This configuration may allow for the top hem 44 or the bottom hem 46 to be stretched to receive or remove a bladder therethrough as described in detailed below.

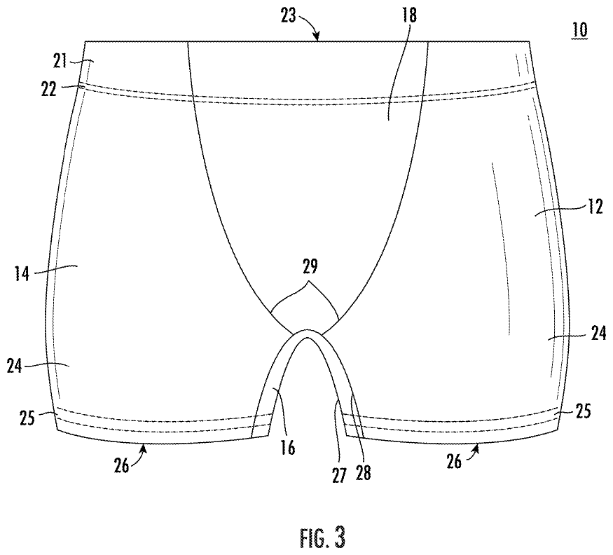

With particular reference to FIG. 2, the middle panel 34 and the inner panel 36 define a hole 38 that is positioned, sized, and dimensioned to allow male genitalia to pass therethrough and be received between the middle and outer panels 32, 34 of the pouch 30. The inner panel 36 may be secured to the middle panel 34 by a seam 52 about the hole 38. The upper and lower edges 54, 56 of the inner panel 36 may include a serge to finish the edges thereof. The inner panel 36 may provide reinforcement about the hole 38 and may smooth edges of the hole 38. In some embodiments, the pouch 30 may not include an inner panel 36 with the middle panel 34 including the hem 52 and defining the hole 38.

Referring now to FIGS. 4-6, the use of a bladder system 100 with the boxer brief 10 is described in accordance with the present disclosure. The bladder system 100 includes a receptacle 120 and a hose 130. The receptacle 120 may be in the form of a cup that is sized and dimensioned to receive genitalia of the pilot such that urine is guided into the hose 130 without contacting skin of the pilot or material of the boxer brief 10. The bladder system 100 may include a reservoir (not shown) that may be secured to the leg of a pilot or be positioned adjacent the pilot wearing the boxer brief 10. The hose 130 has a first end 132 that is coupled to the receptacle 120 and is in communication with an interior of the receptacle 120 and a second end 134 that is configured to secure to the reservoir. The second end 134 may be configured to releasably secure to the reservoir. In some embodiments, the bladder system 100 consists of the receptacle 120 such that the receptacle 120 functions as the reservoir. In some embodiments, the bladder system 100 includes a suction system that is configured to draw fluid, e.g., urine, from the receptacle 120 or the hose 130 into a reservoir of the bladder system 100.

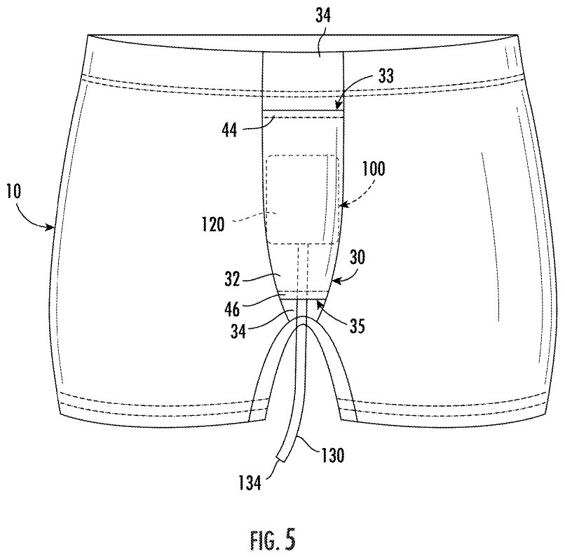

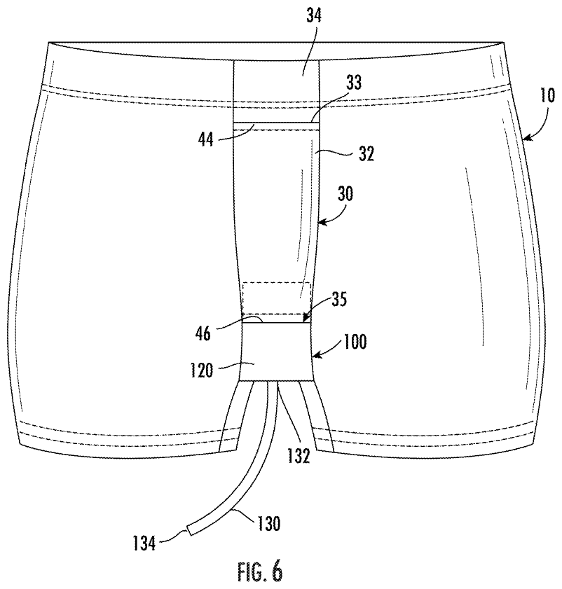

Initially referring to FIG. 4, the second end 134 of the hose 130 is passed through the top pouch opening 33 and the bottom pouch opening 35 such that the hose 130 extends between the outer and middle panels 32, 34 of the pouch 30. With the hose 130 passed through the pouch 30, the receptacle 120 is passed through the top pouch opening 33 such that the receptacle 120 is positioned within the pouch 30 between the outer and middle panels 32, 34 as shown in FIG. 5. As the receptacle 120 passes through the top pouch opening 33, the top hem 44 and the outer panel 32 may be stretched to receive the receptacle 120. The bottom pouch opening 35 is sized to retain the receptacle 120 within the pouch 30.

With the receptacle 120 positioned within the pouch 30, an opening of the receptacle 120 is aligned within the hole 38 (FIG. 2) of the pouch 30 such that genitalia passing through the hole 38 can be received within an interior of the receptacle 120. With the receptacle 120 positioned within the pouch 30 and the genitalia received within the receptacle 120, the second opening 134 of the hose 130 can be secured to a reservoir. The second opening 134 of the hose 130 may be secured to the reservoir before the receptacle 120 is positioned within the pouch 30.

With the receptacle 120 received within the pouch 30 and the hose 134 secured to the reservoir, the outer panel 32 maintains the position of the receptacle 120 within the pouch 30. With the receptacle 120 within the pouch 30, a pilot can utilize the bladder system 100 to urinate without using facilities. The boxer brief 10 maintains the position of the receptacle 120 while preventing leakage and spillage of urine from the bladder system 100 such that the pilot can maintain focus on a task or mission and comfortably relieve himself when necessary.

Referring now to FIG. 6, after the completion of the task or the mission, the pilot can remove the receptacle 120 through the bottom pouch opening 34 of the pouch 30. As the receptacle 120 is removed through the bottom pouch opening 34, the bottom hem 46 and the outer panel 32 may be stretched such that the receptacle 120 can be removed without excessively compressing or folding the receptacle 120. This is in contrast to previous designs were the receptacle 120 was required to be removed through the top opening such that the disconnected second end of a hose, e.g., end 134, could leak or spill contents of the receptacle 120 on garments or the skin of a pilot. Thus, allowing the receptacle 120 to be removed through the bottom opening 35 may prevent or reduce spilling of contents of the receptacle 120, e.g., urine, on garments of the pilot and/or skin of the pilot.

The seams described above may be flat lock seams that are configured to promote comfort and/or reduce irritation when in contact with skin of a pilot wearing the boxer brief 10. The seams may be ⅜ inch seams. In addition, the hems described above may be ¼ inch hems, ⅜ inch hems, or ⅝ inch hems and may include 2NDL cover stitching.

The fabric used to form the panels and the threads used to form the hems and seams may be flame resistant or flame retardant materials. For example, the panels may be formed of a modacrylic, FR rayon, and/or spandex and the thread may be formed of Nomex®. In some embodiments, the entire boxer brief 10 may be flame resistant or flame retardant. For example, the entire boxer brief 10 may be formed of flame resistant or flame retardant materials or may be treated to be flame resistant or flame retardant. In certain embodiments, the fabrics and/or the threads forming the boxer briefs 10 may be resilient or stretchable and in some embodiments, may be considered compression fabric.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A boxer brief comprising:
first and second side panels, each of the first and second side panels having a front edge; and
a pouch comprising:
a first pouch panel having a first side edge attached to the front edge of the first side panel and a second side edge opposite the first side edge attached to the front edge of the second side panel, the first pouch panel defining a hole therethrough, the hole configured to receive genitalia therethrough; and
a second pouch panel having a first side edge attached to the front edge of the first side panel and a second side edge opposite the first side edge attached to the front edge of the second side panel such that the second pouch panel is disposed over the first pouch panel to define a pocket between the first and second side panels, the second pouch panel having a top edge extending between the first and second side edges thereof that is free from the first pouch panel to define a top opening of the pocket and having a bottom edge extending between the first and second side edges thereof that is free from the first pouch panel to define a bottom opening of the pocket opposite the top opening, the pouch configured to secure a bladder system within the pocket, the pocket capable of receiving the bladder system through the top opening and capable of having the bladder system removed through the bottom opening.

2. The boxer brief according to claim 1, wherein the front edge of the first side panel, the first side edge of the first pouch panel, and the first side edge of the second pouch panel are attached to one another along a first pouch seam and the front edge of the second side panel, the second side edge of the first pouch panel, and the second side edge of the second pouch panel are attached to one another along a second pouch seam.

3. The boxer brief according to claim 2, wherein the first and second pouch seams are formed of fire or flame retardant thread.

4. The boxer brief according to claim 1, wherein the first side panel, the second side panel, the first pouch panel, and the second pouch panel are fire or flame retardant.

5. The boxer brief according to claim 1, further comprising a back panel opposite the pouch, the back panel having a first side edge and a second side edge opposite the first side edge, the first side panel having a rear edge attached to the first side edge of the back panel and the second side panel having a rear edge attached to the second side edge of the back panel.

6. The boxer brief according to claim 5, further comprising a bottom panel attached to at least one edge of the first side panel, the second side panel, the back panel, and the first pouch panel.

7. The boxer brief according to claim 5, further comprising a waistband formed by a top portion of the first side panel, the second side panel, the back panel, and the first pouch panel.

8. The boxer brief according to claim 1, wherein the pouch includes a third pouch panel having first side edge attached to the front edge of the first side panel and a second side edge attached to the front edge of the second side panel, the hole passing through the third pouch panel, the third pouch panel attached to the first pouch panel about the hole.

9. The boxer brief according to claim 8, wherein the first pouch panel is disposed between the second and third pouch panels.

10. The boxer brief according to claim 8, wherein the third pouch panel includes top and bottom edges that each extend between the first and second side edges thereof, the top and bottom edges of the third pouch panel free from the first pouch panel.

11. The boxer brief according to claim 1, wherein the second pouch panel is formed of a stretchable material.

12. The boxer brief according to claim 11, wherein the first side panel, the second side panel, and the first pouch panel are formed of a stretchable material.

13. A relief system comprising:
a boxer brief comprising:
first and second side panels, each of the first and second side panels having a front edge; and
a pouch comprising:
a first pouch panel having a first side edge attached to the front edge of the first side panel and a second side edge opposite the first side edge attached to the front edge of the second side panel, the first pouch panel defining a hole therethrough, the hole configured to receive genitalia therethrough; and
a second pouch panel having a first side edge attached to the front edge of the first side panel and a second side edge opposite the first side edge attached to the front edge of the second side panel such that the second pouch panel is disposed over the first pouch panel to define a pocket between the first and second side panels, the second pouch panel having a top edge extending between the first and second side edges thereof that is free from the first pouch panel to define a top opening of the pocket and having a bottom edge extending between the first and second side edges thereof that is free from the first pouch panel to define a bottom opening of the pocket opposite the top opening; and
a bladder system having a receptacle received within the pocket, the bottom opening sized to prevent the receptacle from passing therethrough when the bottom opening is in a relaxed state, the receptacle capable of passing through the top opening and the bottom opening.

14. The relief system according to claim 13, wherein the bladder system including a hose extending from and in fluid communication with the receptacle, the hose passing through the bottom opening of the pocket when the receptacle is received within the pocket.

15. The relief system according to claim 14, wherein the hose has a first end coupled to the receptacle and a second end opposite the first end, the second end configured to releasably couple to a reservoir.

16. A boxer brief comprising:
a waistband defining a waist opening at a top of the boxer brief;
a rear side extending downward from the waistband;
a front side extending downward from the waistband opposite the rear side; and
a pouch on the front side defining a pocket having a top opening adjacent the waistband and a bottom opening opposite the top opening, the pouch configured to retain a receptacle of a bladder system within the pocket, the top opening and the bottom opening capable of allowing the receptacle to pass therethrough.

17. The boxer brief according to claim 16, wherein the pouch comprises an outer panel and an inner panel with the pocket being defined between the inner and outer panels.

18. The boxer brief according to claim 17, wherein the pouch further comprises a middle panel disposed between the inner and outer panels, the inner, middle, and outer panels are attached to one another along pouch seams extending along opposite sides of each of the inner, middle, and outer panels.

19. The boxer brief according to claim 18, wherein the inner and middle panels each define a hole that passes therethrough, the inner and middle panels hemmed together about the hole.

20. The boxer brief according to claim 18, wherein the outer panel has a top edge and a bottom edge that are free from the middle panel to define the top opening and the bottom opening respectively.
